(19) United States
(12) Patent Application Publication
(10) Pub. No.: US 2005/0267202 A1
(43) Pub. Date: Dec. 1, 2005

(54) Chromanol derivatives, a process for their production and their use as anti-inflammatory agents

(75) Inventors: Duy Nguyen, Berlin (DE); Heike Schaecke, Berlin (DE); Konrad Krolikiewicz, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Konrad KROLIKIEWICZ

(21) Appl. No.: 11/130,393

(22) Filed: May 17, 2005

Related U.S. Application Data
(60) Provisional application No. 60/576,408, filed on Jun. 3, 2004.

(30) Foreign Application Priority Data
May 19, 2004 (DE) 102004025791.4

(51) Int. Cl. A61K 31/353

(52) U.S. Cl. 514/456; 549/403

(57) ABSTRACT

The invention relates to compounds of formula I, 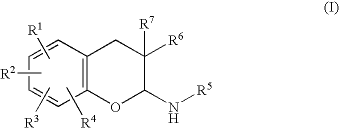 a process for their production, and their use as anti-inflammatory agents.

This application claims the benefit of the filing date of U.S. Provisional Application Ser. No. 60/576,408 filed Jun. 3, 2005 which is incorporated by reference herein

The invention relates to compounds of formula I, a process for their production, and their use as anti-inflammatory agents.

WO 98/45252 and WO 96/15099, whose general formulas also encompass chromanol derivatives, are known from the prior art. An overlapping of the claims with those of this invention does not exist, however.

In both documents, moreover, no chromal derivatives are specifically disclosed. The first-mentioned document relates to an invention in the field of pesticides, and the second-mentioned relates to an invention that contains compounds that are suitable for treating diseases of the central nervous system.

It has now been found, surprisingly enough, that the chromanol derivatives of this invention bind to the glucocorticoid receptors and are suitable as anti-inflammatory agents.

This invention therefore relates to compounds of general formula I 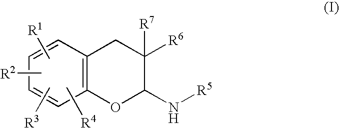
in which
R¹, R² and R³, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a C₁-C₅-perfluoroalkyl group, a cyano group, a nitro group, or a group NR⁸R⁹,
whereby R⁸ and R⁹, independently of one another, can be a hydrogen atom, a (C₁-C₅)-alkyl group, a CO[O(C₁-C₅)]-alkyl group or a (CO)—C₁-C₅-alkyl radical,
R⁴ means a hydrogen atom, a hydroxy group, a halogen atom, a cyano group, an optionally substituted (C₁-C₁₀)-alkyl group, a (C₁-C₁₀)-alkoxy group, or R¹ and R² or R² and R³ or R³ and R⁴ together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—,
whereby n=1 or 2, and the terminal oxygen atoms, nitrogen atoms and/or carbon atoms are linked to directly adjacent ring-carbon atoms,
R⁵ means a C₁-C₁₀-alkyl group, which optionally can be substituted by a group that is selected from 1-3 hydroxy groups, halogen atoms, or 1-3 (C₁-C₅)-alkoxy groups,
an optionally substituted (C₃-C₇)-cycloalkyl group,
an optionally substituted heterocyclyl group,
an optionally substituted aryl group,
a monocyclic or bicyclic aromatic, partially aromatic or non-aromatic ring system, which optionally contains 1-3 nitrogen atoms, 1-2 oxygen atoms and/or 1-2 sulfur atoms and/or 1-2 keto groups, and optionally is substituted in one or more places, independently of one another, by a radical that is selected from the group of carbonyl, halogen atom, hydroxy group, or (C₁-C₅)-alkyl group, which optionally can be substituted by 1-3 hydroxy groups, 1-3 (C₁-C₅)alkoxy groups and/or 1-3 COOR¹⁰ groups, (C₁-C₅)alkoxy group, (C₁-C₅)-alkylthio group, (C₁-C₅)-perfluoralkyl group, cyano group, nitro group, or the radical NR⁸R⁹
whereby R⁸ and R⁹, independently of one another, mean hydrogen, C₁-C₅-alkyl, a CO[O(C₁-C₅)]-alkyl group or (CO)—C₁-C₅-alkyl group,
or the group COOR¹⁰,
whereby R¹⁰ means hydrogen or a C₁-C₅-alkyl group,
or the group (CO)NR¹¹R¹²,
whereby R¹¹ and R¹², independently of one another, mean hydrogen or a C₁-C₅-alkyl group,
or a (C₁-C₅-alkylene)-O—(CO)—(C₁-C₅)alkyl group,
whereby this ring system can be linked via any position to the amine of the chromanol system and optionally can be hydrogenated in one or more places,
R⁶ means a (C₁-C₅)-alkyl group or an optionally partially or completely fluorinated (C₁-C₅)-alkyl group, a (C₃-C₇)cycloalkyl group, a (C₁-C₈)alkyl(C₃-C₇)cycloalkyl group, a (C₂-C₈)alkenyl(C₃-C₇)cycloalkyl group, a heterocyclyl group, a (C₁-C₈)alkylheterocyclyl group, a (C₂-C₈)-alkenylheterocyclyl group, a (C₂-C₈)alkinylaryl group, an aryl group, a (C₁-C₈)alkylaryl group, a (C₂-C₈)alkenylaryl group; a monocyclic or bicyclic heteroaryl group, (C₁-C₈)alkylheteroaryl group or (C₂-C₈)alkenylheteroaryl group that is optionally substituted by 1-2 keto groups, 1-2 (C₁-C₅)-alkyl groups, 1-2 (C₁-C₅)-alkoxy groups, 1-3 halogen atoms, or 1-2 exomethylene groups and/or that contains 1-3 nitrogen atoms and/or 1-2 oxygen atoms and/or 1-2 sulfur atoms,
R⁷ means a hydroxy group or a group OR¹³
whereby R¹³ means a C₁-C₁₀-alkyl group,
   as well as their racemates or separately present stereoisomers, and optionally their physiologically compatible salts.

Compounds of general formula I, in which
R¹, R² and R³, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a C₁-C₅-perfluoroalkyl group, a cyano group, a nitro group, or a group NR⁸R⁹,
whereby R⁸ and R⁹, independently of one another, can be a hydrogen atom, a (C₁-C₅)-alkyl group, a CO[O(C₁-C₅)]-alkyl group or a (CO)—C₁-C₅-alkyl radical,
R⁴ means a hydrogen atom, a hydroxy group, a halogen atom, a cyano group, an optionally substituted (C₁-C₁₀)-alkyl group, a (C₁-C₁₀)-alkoxy group or R¹ and R² or R² and R³ or R³ and R⁴ together mean a group that is selected from the groups —O—(CH₂)ₙ—O—, —O(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—,
whereby n=1 or 2, and the terminal oxygen atoms, nitrogen atoms and/or carbon atoms are linked to directly adjacent ring-carbon atoms,
R⁵ means a monocyclic or bicyclic aromatic, partially aromatic or non-aromatic ring system, which optionally contains 1-3 nitrogen atoms, 1-2 oxygen atoms, and/or 1-2 sulfur atoms and/or 1-2 keto groups, and optionally is substituted in one or more places, independently of one another, by a radical that is selected from the group of carbonyl, halogen atom, hydroxy group, (C₁-C₅)-alkyl group, which optionally can be substituted by 1-3 hydroxy groups, 1-3 (C₁-C₅)alkoxy groups and/or 1-3 COOR¹⁰ groups,
(C₁-C₅)alkoxy group, (C₁-C₅)-alkylthio group, (C₁-C₅)-perfluoroalkyl group, cyano group, nitro group, or the radical NR⁸R⁹,
whereby R⁸ and R⁹, independently of one another, mean hydrogen, C₁-C₅-alkyl, a CO[O(C₁-C₅)]-alkyl group or (CO)-C₁-C₅-alkyl group,
or the group COOR¹⁰,
whereby R¹⁰ means hydrogen or a C₁-C₅-alkyl group,
or the group (CO)NR¹¹R¹²,
whereby R¹¹ and R¹², independently of one another, mean hydrogen or a C₁-C₅-alkyl group,
or a (C₁-C₅-alkylene)-O—(CO)—(C₁-C₅)alkyl group,
whereby this ring system can be linked via any position with the amine of the chromanol system and optionally can be hydrogenated at one or more locations,
R⁶ means a (C₁-C₅)-alkyl group or an optionally partially or completely fluorinated (C₁-C₅)-alkyl group, a (C₃-C₇)cycloalkyl group, a (C₁-C₈)alkyl(C₃-C₇)cycloalkyl group, a (C₂-C₈)alkenyl(C₃-C₇)cycloalkyl group, a heterocyclyl group, a (C₁-C₈)alkylheterocyclyl group, a (C₂-C₈)-alkenylheterocyclyl group, a (C₂-C₈)alkinylaryl group, an aryl group, a (C₁-C₈)alkylaryl group, a (C₂-C₈)alkenylaryl group, a monocyclic or bicyclic heteroaryl group, (C₁-C₈)alkylheteroaryl group or (C₂-C₈)alkenylheteroaryl group that is optionally substituted by 1-2 keto groups, 1-2 (C₁-C₅)-alkyl groups, 1-2 (C₁-C₅)-alkoxy groups, 1-3 halogen atoms, or 1-2 exomethylene groups and/or that contains 1-3 nitrogen atoms and/or 1-2 oxygen atoms and/or 1-2 sulfur atoms,
R⁷ means a hydroxy group or a group OR¹³,
whereby R¹³ means a C₁-C₁₀-alkyl group,
   as well as their racemates or separately present stereoisomers, and optionally their physiologically compatible salts are another subject of the invention.

Compounds of general formula I, in which
R¹, R² and R³, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a C₁-C₅-perfluoralkyl group, a cyano group, a nitro group, or a group NR⁸R⁹,
whereby R⁸ and R⁹, independently of one another, can be a hydrogen atom, a (C₁-C₅)-alkyl group, a CO[O(C₁-C₅)]-alkyl group or a (CO)-C₁-C₅-alkyl radical,
R⁴ means a hydrogen atom, a hydroxy group, a halogen atom, a cyano group, an optionally substituted (C₁-C₁₀)-alkyl group, a (C₁-C₁₀)-alkoxy group or R¹ and R² or R¹ and R¹ or R³ and R¹ together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, —NH—N═CH—,
whereby n=1 or 2, and the terminal oxygen atoms, nitrogen atoms and/or carbon atoms are linked to directly adjacent ring-carbon atoms,
R⁵ means a monocyclic or bicyclic aromatic, partially aromatic or non-aromatic ring system, which optionally contains 1-3 nitrogen atoms, 1-2 oxygen atoms and/or 1-2 sulfur atoms and/or 1-2 keto groups, and optionally is substituted in one or more places, independently of one another, by a radical that is selected from the group of carbonyl, halogen atom, hydroxy group, (C₁-C₅)-alkyl group, which optionally can be substituted by 1-3 hydroxy groups, 1-3 (C₁-C₅)alkoxy groups and/or 1-3 COOR¹⁰ groups,
(C₁-C₅)alkoxy group, (C₁-C₅)-alkylthio group, (C₁-C₅)-perfluoroalkyl group, cyano group, nitro group, or the radical NR⁸R⁹,
whereby R⁸ and R⁹, independently of one another, mean hydrogen, C₁-C₅-alkyl, a CO[O(C₁-C₅)]-alkyl group or a (CO)—C₁-C₅-alkyl group,
or the group COOR¹⁰,
whereby R¹⁰ means hydrogen or a C₁-C₅-alkyl group,
or the group (CO)NR¹¹R¹²,
whereby R¹¹ and R², independently of one another, mean hydrogen or a C₁-C₅-alkyl group,
or a (C₁-C₅-alkylene)-O—(CO)—(C₁-C₅)alkyl group,
whereby this ring system can be linked via any position to the amine of the chromanol system and optionally can be hydrogenated on one or more locations,
R⁶ means an optionally partially or completely fluorinated (C₁-C₅)-alkyl group,
R⁷ means a hydroxy group or a group OR¹³,
whereby R¹³ means a C₁-C₁₀-alkyl group,
   as well as their racemates or separately present stereoisomers, and optionally their physiologically compatible salts are another subject of the invention.

Compounds of general formula I, in which

R¹, R², R³, R⁴, independently of one another, mean hydrogen, C₁-C₅-alkyl, C₁-C₅-alkoxy, C₁-C₅-alkylthio, C₁-C₅-perfluoroalkyl, halogen, hydroxy, cyano, or nitro

are another subject of the invention.

Compounds of general formula I in which R¹/R² or R²/R³ or R³/R⁴ mean —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—, whereby n=1 or 2 and the terminal oxygen atoms, nitrogen atoms and/or carbon atoms are linked to directly adjacent ring-carbon atoms, are another aspect of the invention.

Compounds of general formula I in which R⁶ means a (C₁-C₃)-alkyl group or an optionally partially or completely fluorinated (C₁-C₃)-alkyl group are a special subject of the invention. Preferred is the completely fluroinated (C₁-C₃)-alkyl group. Especially preferred are compounds of general formula I, in which R⁶ stands for a trifluoromethyl or pentafluoroethyl radical.

Racemates or separately present stereoisomers, and optionally physiologically compatible salts of all subjects of this invention are another aspect of this invention.

Compounds of general formula I in which R⁵ means an optionally substituted phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that is linked via any position as well as their physiologically compatible salts are another subject of the invention.

Compounds of general formula I in which R⁵ means a phthalidyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that is linked via any position are another subject of the invention.

The nitrogen atom in indazole, quinolone, isoquinolone and phthalazine of general claim 1 can also be alkylated with a C₁-C₃-alkyl group.

Compounds of formula (I), in which R⁵ means a phthalidyl, isoindolyl, dihydroindolyl, dihydroisoindolyl, thiophthalidyl, indazolyl, benzothiazolyl, dihydroindolonyl, dihydroisoindolonyl, benzimidazolyl or indolyl group are another subject of the invention.

Compounds of formula (I) in which R⁵ means dihydroisoquinolinyl, dihydroquinolinyl, benzoxazinonyl, phthalazinonyl, quinolinyl, isoquinolinyl, quinolonyl, isoquinolonyl, quinazolinyl, quinoxalinyl, cinnolinyl, phthalazinyl, 1,7- or 1,8-naphthyridinyl are another subject of the invention.

Compounds of general formula I in which R⁵ means an isoquinolonyl, quinolonyl, quinazolinyl or phthalazinyl group are another subject of the invention.

Radical R⁵ of the compounds of general formula I can be substituted by one or more substituents selected from the group of (C₁-C₅)-alkyl group, (C₁-C₅)-alkoxy group, halogen atom, a keto-oxygen atom or a hydroxy group.

Compounds of general formula I, in which R⁵ means a phenyl ring, which optionally can be substituted in one or more places by a radical that is selected from the group C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, —NH—N═CH—,

whereby n=1 or 2 and the terminal oxygen atoms, nitrogen atoms and/or carbon atoms are linked to directly adjacent ring-carbon atoms,

or NR⁸R⁹,

whereby R⁸ and R⁹, independently of one another, can be a hydrogen atom, a C₁-C₅-alkyl group or a (CO)—C₁-C₅-alkyl group,

are another aspect of the invention.

With a partially aromatic ring system, bicyclic systems that contain an aromatic and a non-aromatic ring, such as, e.g., benzodihydrofuran, benzodihydrothiophene, benzoxazinone or dihydroindolone, are meant.

In addition, the invention relates to the use of the compounds of general formula I for the production of pharmaceutical agents as well as their use for the production of pharmaceutical agents for treating inflammatory diseases.

The C₁-C₁₀-alkyl groups can be straight-chain or branched and stand for a methyl, ethyl, n-propyl, iso-propyl, n-butyl, iso-butyl, tert.-butyl or n-pentyl, 2,2-dimethylpropyl, 2-methylbutyl or 3-methylbutyl group, a pentyl, an isopentyl, a hexyl, a heptyl, a nonyl or a decyl group. C₁-C₅-Alkyl groups are preferred. A methyl or ethyl group is especially preferred.

They can optionally be substituted by 1-3 hydroxy and/or 1-3 COOR¹⁰ groups, halogen atoms or 1-3 (C₁-C₅)-alkoxy groups.

The C₁-C₁₀-alkoxy groups can be straight-chain or branched and, for example, stand for a methoxy, ethoxy, n-propoxy, iso-propoxy, n-butoxy, iso-butoxy, tert.-butoxy or n-pentoxy, 2,2-dimethylpropoxy, 2-methylbutoxy or 3-methylbutoxy group, a pentyloxy, an isopentyloxy, or a hexyloxy group. C₁-C₅-Alkoxy groups are preferred. The methoxy and ethoxy groups are especially preferred.

The C₁-C₁₀-alkylthio groups can be straight-chain or branched and stand for, for example, a methylthio, ethylthio, n-propylthio, iso-propylthio, n-butylthio, iso-butylthio, tert.-butylthio or n-pentylthio, 2,2-dimethylpropylthio, 2-methylbutylthio or 3-methylbutylthio group. C₁-C₅-Alkylthio groups are preferred. A methylthio or ethylthio group is especially preferred.

For a partially or completely fluorinated C₁-C₁₀-alkyl group, the following partially or completely fluorinated groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, or pentafluoroethyl. The partially or completely fluorinated C₁-C₅-alkyl groups are preferred.

Of the latter, the trifluoromethyl group or the pentafluoroethyl group is especially preferred for radical R⁶.

The completely fluorinated alkyl groups are also referred to as perfluoroalkyl groups.

The designation halogen atom or halogen means a fluorine, chlorine, bromine or iodine atom. A fluorine, chlorine or bromine atom is preferred.

The NR⁸R⁹ group can mean, for example, NH₂, N(H)CH₃, N(CH₃)₂, N(H)(CO)CH₃, N(H)(CO)CH₂CH₃, N(CH₃)(CO)CH₃, N[(CO)CH₃]₂, N(H)CO₂CH₃, N(H)CO₂CH₂CH₃, N(CH₃)CO₂CH₃, N(CH₃)CO₂CH₂CH₃, or N(CO₂CH₃)₂.

The designation (C₃-C₇)-cycloalkyl or non-aromatic ring system means cyclopropyl, cyclobutyl, cyclopentyl, cyclohexyl as well as cycloheptyl. The cycloalkyl groups can be substituted just as for the aryl radical described below.

Heterocyclyl groups are cycloalkyl groups that in addition contain one or more heteroatoms from the group of sulfur, nitrogen, or oxygen, such as, for example, but not exclusively limited to, pyrrolidinyl, piperidinyl, piperazinyl, aziridinyl, tetrahydrofuranyl as well as tetrahydropyranyl, thiomorpholinyl, morpholinyl, and dioxanyl. The heterocyclyl groups can be substituted as described below for the aryl radical.

The designation aryl means phenyl or naphthyl. Phenyl is preferred. As substituents, all substituents that are commonly used for aryl systems are considered, for example, halogen, (C₁-C₅)-alkyl, hydroxy, (C₁-C₅)-alkoxy, (C₁-C₅)-alkylthio, carbonyl, cyano, nitro, NR⁸R⁹, and COOR¹⁰.

The designation heteroaryl means an aromatic, 5- to 8-membered monocyclic ring or an aromatic, 8- to 11-membered bicyclic ring that contains 1-4 heteroatoms from the group of sulfur, nitrogen, and oxygen. This designation relates to, but is not exclusively limited to, thienyl, furanyl, isoxazolyl, thiazolyl, pyrazolyl, pyrrolyl, imidazolyl, pyridinyl, pyrimidinyl, pyrazinyl, pyridazinyl, pyranyl, quinoxalinyl, indolyl, isoindolyl, dihydroindolyl, dihydroisoindolyl, benzimidazolyl, benzoxazolyl, benzothiazolyl, benzothienyl, quinolinyl, dihydroquinolinyl, indazolyl, and indazolinyl.

The aromatic heteroaryl systems can be substituted by one or more substituents that are selected from the group (C₁-C₅)-alkyl group, (C₁-C₅)-alkoxy group, halogen atom or hydroxy group. By way of example, dihydroindolonyl, dihydroisoindolonyl, benzoxazinonyl, phthalidyl, and thiophthalidyl can be mentioned here.

The (C₁-C₈)alkylheteroaryl group or the (C₂-C₈)alkenylheteroaryl group R⁶ can be substituted by the same substituents, the monocyclic or bicyclic heteroaryl group cited directly above in claim 1.

The compounds of general formula I according to the invention can be present as stereoisomers because of the presence of asymmetry centers. Subjects of this invention are all possible diastereomers, both as racemates and in enantiomer-pure form.

The compounds according to the invention can also be present in the form of salts with physiologically compatible anions, for example in the form of hydrochlorides, sulfates, nitrates, phosphates, pivalates, maleates, fumarates, tartrates, benzoates, mesylates, citrates or succinates.

The compounds can be produced by the process described below.

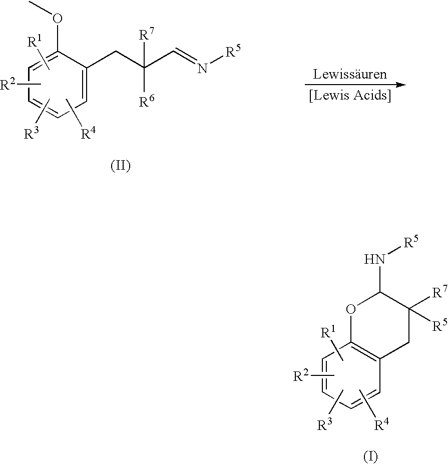

The compounds of formula I are produced in the reaction of the corresponding imines with Lewis acids, preferably in the reaction with boron tribromide. The reaction is carried out in a temperature range of −70° C. to +30° C. (preferably −30° C. to +30° C.).

As Lewis acids, the compounds that are known to one skilled in the art are suitable.

In turn, the imines are formed by the correspondingly described aldehydes being reacted with the desired amines. The latter can be done, as known to one skilled in the art, for example by reaction with titanates or by reaction in an acid medium, for example by stirring at room temperature or by boiling in a water separator.

The acid medium can be achieved by adding inorganic or organic acids, for example hydrochloric acid, sulfuric acid or acetic acid.

The binding of substances to the glucocorticoid receptor (GR) and other steroid hormone receptors (mineral corticoid receptor (MR), progesterone receptor (PR) and androgen receptor (AR)) is examined with the aid of recombinantly produced receptors. Cytosol preparations of Sf9 cells, which had been infected with recombinant baculoviruses, which code for the GR, are used for the binding studies. In comparison to reference substance [³H]-dexamethasone, the substances show a high to very high affinity to GR.

As an essential, molecular mechanism for the anti-inflammatory action of glucocorticoids, the GR-mediated inhibition of the transcription of cytokines, adhesion molecules, enzymes and other pro-inflammatory factors is considered. This inhibition is produced by an interaction of the GR with other transcription factors, e.g., AP-1 and NF-kappa-B (for a survey, see Cato, A. C. B., and Wade, E., BioEssays 18, 371-378, 1996).

The compounds of general formula I according to the invention inhibit the secretion of cytokine IL-8 into the human monocyte cell line THP-1 that is triggered by lipopolysaccharide (LPS). The concentration of the cytokines was determined in the supernatant by means of commercially available ELISA kits.

The anti-inflammatory action of the compounds of general formula I was tested in the animal experiment by tests in the croton oil-induced inflammation in rats and mice (J. Exp. Med. (1995), 182, 99-108). To this end, croton oil in ethanolic solution was applied topically to the animals' ears. The test substances were also applied topically or systemically at the same time or two hours before the croton oil. After 16-24 hours, the ear weight was measured as a yardstick for inflammatory edema, the peroxidase activity as a yardstick for the invasions of granulocytes, and the elastase activity as a yardstick for the invasion of neutrophilic granulocytes. In this test, the compounds of general formula I inhibit the three above-mentioned inflammation parameters both after topical administration and after systemic administration.

One of the most frequent undesirable actions of a glucocorticoid therapy is the so-called “steroid diabetes” [cf., Hatz, H. J., Glucocorticoide: Immunologische Grundlagen, Pharmakologie und Therapierichtlinien, [Glucocorticoids: Immunological Bases, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). A key enzyme of the catabolic metabolism in the liver is tyrosinamino transferase (TAT). The activity of this enzyme can be determined from liver homogenates by photometry and represents a good measurement of the undesirable metabolic actions of glucocorticoids. To measure the TAT induction, the animals are sacrificed 8 hours after the test substances are administered, the livers are removed, and the TAT activity is measured in the homogenate. In this test, at doses in which they have an anti-inflammatory action, the compounds of general formula I induce little or no tyrosinamino transferase.

Because of their anti-inflammatory and additional anti-allergic, immunosuppressive and antiproliferative action, the compounds of general formula I according to the invention can be used as medications for treatment or prophylaxis of the following pathologic conditions in mammals and humans: In this case, the term “DISEASE” stands for the following indications:
(i) Lung diseases that are accompanied by inflammatory, allergic and/or proliferative processes:
   Chronic, obstructive lung diseases of any origin, primarily bronchial asthma
   Bronchitis of different origins
   All forms of restrictive lung diseases, primarily allergic alveolitis,
   All forms of pulmonary edema, primarily toxic pulmonary edema
   Sarcoidoses and granulomatoses, especially Boeck's disease
(ii) Rheumatic diseases/autoimmune diseases/joint diseases that are accompanied by inflammatory, allergic and/or proliferative processes:
   All forms of rheumatic diseases, especially rheumatoid arthritis, acute rheumatic fever, polymyalgia rheumatica
   Reactive arthritis
   Inflammatory soft-tissue diseases of other origins
   Arthritic symptoms in the case of degenerative joint diseases (arthroses)
   Traumatic arthritides
   Collagenoses of any origin, e.g., systemic lupus erythematodes, sclerodermia, polymyositis, dermatomyositis, Sjögren's syndrome, Still's syndrome, Felty's syndrome
(iii) Allergies that are accompanied by inflammatory and/or proliferative processes:
   All forms of allergic reactions, e.g., Quincke's edema, hay fever, insect bites, allergic reactions to pharmaceutical agents, blood derivatives, contrast media, etc., anaphylactic shock, urticaria, contact dermatitis
(iv) Vascular inflammations (vasculitides)
   Panarteritis nodosa, arteritis temperalis, erythema nodosum
(v) Dermatological diseases that are accompanied by inflammatory, allergic and/or proliferative processes:
   Atopic dermatitis (primarily in children)
   Psoriasis
   Pityriasis rubra pilaris
   Erythematous diseases, triggered by different noxae, e.g., radiation, chemicals, burns, etc.
   Bullous dermatoses
   Diseases of the lichenoid group,
   Pruritis (e.g., of allergic origin)
   Seborrheal eczema
   Rosacea
   Pemphigus vulgaris
   Erythema exudativum multiforme
   Balanitis
   Vulvitis
   Hair loss such as alopecia greata
   Cutaneous T-cell lymphoma
(vi) Kidney diseases that are accompanied by inflammatory, allergic and/or proliferative processes:
   Nephrotic syndrome
   All nephritides
(vii) Liver diseases that are accompanied by inflammatory, allergic and/or proliferative processes:
   Acute liver cell decomposition
   Acute hepatitis of different origins, e.g., viral, toxic, pharmaceutical agent-induced
   Chronic aggressive hepatitis and/or chronic intermittent hepatitis
(viii) Gastrointestinal diseases that are accompanied by inflammatory, allergic and/or proliferative processes:
   Regional enteritis (Crohn's disease)
   Colitis ulcerosa
   Gastritis
   Reflux esophagitis
   Ulcerative colitis of other origins, e.g., native sprue
(ix) Proctologic diseases that are accompanied by inflammatory, allergic and/or proliferative processes:
   Anal eczema
   Fissures
   Hemorrhoids
   Idiopathic proctitis
(x) Eye diseases that are accompanied by inflammatory, allergic and/or proliferative processes:
   Allergic keratitis, uveitis, iritis
   Conjunctivitis
   Blepharitis
   Optic neuritis
   Chorioiditis
   Sympathetic ophthalmia
(xi) Diseases of the ear-nose-throat area that are accompanied by inflammatory, allergic and/or proliferative processes:
   Allergic rhinitis, hay fever
   Otitis extema, e.g., caused by contact dermatitis, infection, etc.
   Otitis media
(xii) Neurological diseases that are accompanied by inflammatory, allergic and/or proliferative processes:
   Cerebral edema, primarily tumor-induced cerebral edema
   Multiple sclerosis
   Acute encephalomyelitis
   Meningitis
   Various forms of convulsions, e.g., infantile nodding spasms
(xiii) Blood diseases that are accompanied by inflammatory, allergic and/or proliferative processes:
   Acquired hemolytic anemia
   Idiopathic thrombocytopenia
(xiv) Tumor diseases that are accompanied by inflammatory, allergic and/or proliferative processes:
   Acute lymphatic leukemia
   Malignant lymphoma
   Lymphogranulomatoses
   Lymphosarcoma
   Extensive metastases, mainly in breast, bronchial and prostate cancers
(xv) Endocrine diseases that are accompanied by inflammatory, allergic and/or proliferative processes:
   Endocrine orbitopathy
   Thyreotoxic crisis
   De Quervain's thyroiditis
   Hashimoto's thyroiditis
   Basedow's disease
(xvi) Organ and tissue transplants, graft-versus-host disease
(xvii) Severe shock conditions, e.g., anaphylactic shock, systemic inflammatory response syndrome (SIRS)
(xviii) Substitution therapy in:
   Innate primary suprarenal insufficiency, e.g., congenital adrenogenital syndrome
   Acquired primary suprarenal insufficiency, e.g., Addison's disease, autoimmune adrenalitis, meta-infective tumors, metastases, etc.
   Innate secondary suprarenal insufficiency, e.g., congenital hypopituitarism
   Acquired secondary suprarenal insufficiency, e.g., meta-infective tumors, etc.
(xix) Vomiting that is accompanied by inflammatory, allergic and/or proliferative processes:
   e.g., in combination with a 5-HT3 antagonist in cytostatic-agent-induced vomiting
(xx) Pains of inflammatory origins, e.g., lumbago.

Moreover, the compounds of general formula I according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

All previously mentioned indications (i) to (xx) are described in more detail in Hatz, H. J., Glucocorticoide: Immunologische Grundlagen, Pharmakologie und Therapierichtlinien, Wissenschaftliche Verlagsgesellschaft mbH, Stuttgart, 1998.

For the therapeutic actions in the above-mentioned pathologic conditions, the suitable dose varies and depends on, for example, the active strength of the compound of general formula I, the host, the type of administration, and the type and severity of the conditions that are to be treated, as well as the use as a prophylactic agent or therapeutic agent.

The invention additionally provides:
(i) The use of one of the compounds of general formula I according to the invention or mixture thereof for the production of a medication for treating a DISEASE;
(ii) A process for treating a DISEASE, said process comprises an administration of an amount of the compound according to the invention, whereby the amount suppresses the disease and whereby the amount of compound is given to a patient who requires such a medication;
(iii) A pharmaceutical composition for treating a DISEASE, said treatment comprises one of the compounds according to the invention or mixture thereof and at least one pharmaceutical adjuvant and/or vehicle.

In general, satisfactory results can be expected in animals when the daily doses comprise a range of 1 μg to 100,000 μg of the compound according to the invention per kg of body weight. In the case of larger mammals, for example the human, a recommended daily dose lies in the range of 1 μg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. For treating acute shock (e.g., anaphylactic shock), individual doses can be given that are significantly above the above-mentioned doses.

The formulation of the pharmaceutical preparations based on the new compounds is carried out in a way that is known in the art by the active ingredient being processed with the vehicles that are commonly used in galenicals, fillers, substances that influence decomposition, binding agents, moisturizers, lubricants, absorbents, diluents, flavoring correctives, coloring agents, etc., and converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments and tinctures in corresponding pharmaceutical preparations.

For topical application, formulations in gels, ointments, fatty ointments, creams, pastes, powders, milk and tinctures are possible. The dosage of the compounds of general formula I should be 0.01%-20% in these preparations to achieve a sufficient pharmacological action.

The invention also comprises the compounds of general formula I according to the invention as therapeutic active ingredients. In addition, the compounds of general formula I according to the invention are part of the invention as therapeutic active ingredients together with pharmaceutically compatible and acceptable adjuvants and vehicles.

The invention also comprises a pharmaceutical composition that contains one of the pharmaceutically active compounds according to the invention or mixtures thereof or a pharmaceutically compatible salt thereof and a pharmaceutically compatible salt or pharmaceutically compatible adjuvants and vehicles.

### EXPERIMENTAL SECTION

### Example 1

### 5-(3-Hydroxy-3-trifluoromethyl-chroman-2-ylamino)-1H-quinolin-2-one

### a) 3,3,3-Trifluoro-2-hydroxy-2-(2-methoxybenzyl)-propionaldehyde

5.4 g (24.75 mmol) of 1,1,1-trifluoro-3-(2-methoxyphenyl)-propan-2-one (for preparation, cf. J. Boivin, L. R. Kaim, S. Z. Zard, Tetrahedron Letters 1992, 33, 1285-1288) is dissolved in tetrahydrofuran (60 ml) and water (20 ml) and mixed with a solution that consists of potassium cyanide (1.86 g, 28.5 mmol) in water (20 ml) at 0° C. After 10 minutes, 10 ml of sulfuric acid (25%) is added at the same temperature. It is stirred for 20 minutes at 0° C., then for 20 hours at room temperature. The reaction is completed by adding saturated sodium bicarbonate solution. It is extracted with ethyl acetate. The combined organic phases are washed with saturated sodium chloride solution and dried on sodium sulfate. After the solvent is removed under reduced pressure, 5.94 g (98% of theory) of 3,3,3-trifluoro-2-hydroxy-2-(2-methoxybenzyl)-propionitrile, which is further reacted without purification, is obtained.

A solution that consists of 3,3,3-trifluoro-2-hydroxy-2-(2-methoxybenzyl)-propionitrile (1.23 g, 5.02 mmol) in diethyl ether (30 ml) is mixed at −80° C. with 8.8 ml of a 1.2 M diisobutyl aluminum hydride solution in toluene. It is stirred for one hour at −80° C., and then for 3 hours at room temperature. 50 ml of a 10% aqueous tartaric acid solution is added at 0° C., and the reaction mixture is allowed to stir for one hour at room temperature. Then, the aqueous phase is separated and extracted with diethyl ether. The combined organic phases are washed with saturated sodium chloride solution and dried on sodium sulfate. After the solvent is removed and after chromatographic purification (silica gel, hexane/ethyl acetate (0-10% ethyl acetate) of the residue, 0.453 g (36% of theory) of the product is obtained.

NMR (300 MHz, DMSO-d₆): δ2.95 (d, 1H), 2.36 (d, 1H), 3.72 (s, 3H), 6.86-6.96 (m, 3H), 7.22-7.28 (m, 2H), 9.57 (s, 1H).

### b) 5-[3,3,3-Trifluoro-2-hydroxy-2-(2-methoxybenzyl)-propylidenamino]-H-quinolin-2-one

3,3,3-Trifluoro-2-hydroxy-2-(2-methoxybenzyl)-propionaldehyde (103 mg, 0.42 mmol) as well as 5-amino-1H-quinolin-2-one (100 mg, 1.5 equivalents) are introduced into 6 ml of xylene and mixed with 0.18 ml (2.0 equivalents) of titanium(IV)ethylate. The reaction mixture is allowed to stir for 3 hours at 150° C. and then to cool off to room temperature. After saturated sodium chloride solution and ethyl acetate are added, it is vigorously stirred for another 30 minutes at room temperature. The precipitate that is produced is suctioned off on Celite and washed with ethyl acetate. The aqueous phase is separated, and the organic phase is dried on sodium sulfate. After the solvent is removed under reduced pressure and chromatographic purification (silica gel, dichloromethane/methanol (0-10% methanol) of the residue, the desired product is obtained in a quantitative yield.

NMR (300 MHz, DMSO-d₆): δ3.00 (d, 1H), 3.50 (d, 1H), 3.61 (s, 3H), 6.38 (d, 1H), 6.48-6.50 (m, 2H), 6.88-6.92 (m, 2H), 7.15 (d, 1H), 7.25 (td, 1H), 7.31 (dd, 1H), 7.45 (t, 1H), 7.59 (d, 1H), 7.97 (s, 1H), 11.78 (s, 1H).

### c) 5-(3-Hydroxy-3-trifluoromethylchroman-2-ylamino)-1H-quinolin-2-one

A solution that consists of 5-[3,3,3-trifluoro-2-hydroxy-2-(2-methoxybenzyl)-propylidenamino]-1H-quinolin-2-one (82 mg, 0.21 mmol) in 2.0 ml of dichloromethane is mixed at room temperature with 2.2 ml (10 equivalents) of a 1 M solution of boron tribromide in dichloromethane. Then, the reaction mixture is allowed to stir for 90 minutes at room temperature. The reaction is completed by the addition of saturated sodium bicarbonate solution at −30° C. and ethyl acetate. After the phases are separated, the aqueous phase is extracted with ethyl acetate. The combined organic phases are dried on sodium sulfate. After the solvent is removed under reduced pressure and after purification of the residue by means of preparative thin-layer chromatography (dichloromethane/methanol 9:1), 67 mg (85% of theory) of the product is obtained.

NMR (300 MHz, DMSO-d₆): δ3.06 (d, 1H), 3.34 (d, 1H), 5.81 (d, 1H), 6.46 (d, 1H), 6.56 (d, 1H), 6.76-6.82 (m, 4H), 6.92 (t, 1H), 7.13 (t, 1H), 7.20 (d, 1H), 7.33 (t, 1H), 8.09 (d, 1H), 11.64 (s, 1H).

### Example 2

### 2-(7-Fluoro-2-methylquinazolin-5-ylamino)-3-trifluoromethyl-chroman-3-ol

### a) 1,1,1-Trifluoro-3-(7-fluoro-2-methylquinazolin-5-ylimino)-2-(2-methoxybenzyl)-propan-2-ol

Analogously to Example 1b), 3,3,3-trifluoro-2-hydroxy-2-(2-methoxybenzyl)-propionaldehyde (248 mg, 1.0 mmol) is reacted with 7-fluoro-2-methylquinazolin-5-ylamine (265 mg, 1.5 mmol) in 15 ml of xylene in the presence of titanium(IV)ethylate (0.42 ml, 2.0 mmol) for 7 hours at 150° C. After working-up and chromatographic purification (silica gel, hexane/ethyl acetate (30-50% ethyl acetate)), 169 mg (41% of theory) of the product is obtained.

NMR (300 MHz, DMSO-d₆): δ2.75 (s, 3H), 3.06 (d, 1H), 3.52 (d, 1H), 3.57 (s, 3H), 6.65 (s, 1H), 6.85-6.94 (m, 3H), 7.24 (t, 1H), 7.32 (d, 1H), 7.53 (dd, 1H), 8.25 (s, 1H), 9.16 (s, 1H).

### b) 2-(7-Fluoro-2-methylquinazolin-5-ylamino)-3-trifluoromethylchroman-3-ol

Analogously to Example 1, a solution that consists of 1,1,1-trifluoro-3-(7-fluoro-2-methyl-quinazolin-5-ylimino)-2-(2-methoxybenzyl)-propan-2-ol (101 mg, 0.248 mmol) in 2.0 ml of dichloromethane is mixed at room temperature with 2.5 ml (10 equivalents) of a 1 M solution of boron tribromide in dichloromethane. After 2 hours, as described in Example 1, it is worked up, and the residue is purified by means of preparative thin-layer chromatography (hexane/ethyl acetate 1:2). 76 mg (78% of theory) of the product is obtained.

NMR (300 MHz, DMSO-d₆): Mixture of the diastereomers, δ2.68 (s, 3H), 2.72 (s, 3H), 2.97 (d, 1H), 3.13 (d, 1H), 3.35 (d, 1H), 3.87 (d, 1H), 5.67 (d, 1H), 6.03 (d, 1H), 6.85-7.26 (m), 7.48 (d, 1H), 7.59 (d, 1H), 9.52 (s, 1H), 9.61 (s, 1H).

### Example 3

### 5-(3-Hydroxy-3-trifluoromethyl-chroman-2-ylamino)-2H-isoquinolin-1-one

### a) 5-[3,3,3-Trifluoro-2-hydroxy-2-(2-methoxybenzyl)-propylidenamino]-2H-iso-quinolin-1-one

Analogously to Example 1, 3,3,3-trifluoro-2-hydroxy-2-(2-methoxybenzyl)-propionaldehyde (248 mg, 1.0 mmol) is reacted with 5-amino-2H-isoquinolin-1-one (240 mg, 1.5 mmol) in 15 ml of xylene in the presence of titanium(IV)ethylate (0.42 ml, 2.0 mmol) for 15 hours at 150° C. After working-up and chromatographic purification (silica gel, ethyl acetate/methanol (0-10% methanol)), 159 mg (41% of theory) of the product is obtained.

NMR (300 MHz, DMSO-d₆): δ3.03 (d, 1H), 3.50 (d, 1H), 3.63 (s, 3H), 6.35 (d, 1H), 6.47 (s, 1H), 6.87-6.93 (m, 2H), 6.98 (d, 1H), 7.09 (t, 1H), 7.27 (t, 1H), 7.32 (d, 1H), 7.44 (t, 1H), 7.96 (s, 1H), 8.04 (d, 1H), 11.35 (s, 1H).

### b) 5-(3-Hydroxy-3-trifluoromethylchroman-2-ylamino)-2H-isoquinolin-1-one

Analogously to Example 1, a solution that consists of 5-[3,3,3-trifluoro-2-hydroxy-2-(2-methoxybenzyl)-propylidenamino]-2H-isoquinolin-1-one (103 mg, 0.264 mmol) in 3.0 ml of dichloromethane is mixed at room temperature with 2.8 ml (10 equivalents) of a 1 M solution of boron tribromide in dichloromethane. After 2 hours, as described in Example 1, it is worked up, and the residue is purified by means of the preparative thin-layer chromatography (hexane/ethyl acetate 3:7). 60 mg (60% of theory) of the product is obtained.

NMR (300 MHz, DMSO-d₆): δ3.04 (d, 1H), 3.35 (d, 1H), 5.86 (d, 1H), 6.28 (d, 1H), 6.68 (d, 1H), 6.82 (d, 1H), 6.90-6.94 (m, 2H), 7.14 (t, 1H), 7.19-7.23 (m, 2H), 7.30-7.37 (m, 2H), 7.69 (d, 1H), 11.29 (d, 1H).

### Example 4

### 2-(8-Fluoro-2-methylquinazolin-5-ylamino)-3-trifluoromethylchroman-3-ol

### a) 1,1,1-Trifluoro-3-(8-fluoro-2-methylquinazolin-5-ylimino)-2-(2-methoxybenzyl)-propan-2-ol

Analogously to Example 1, 3,3,3-trifluoro-2-hydroxy-2-(2-methoxybenzyl)-propionaldehyde (372 mg, 1.49 mmol) is reacted with 8-fluoro-2-methylquinazolin-5-ylamine (398 mg, 2.25 mmol) in 15 ml of xylene in the presence of titanium(IV) ethylate (0.63 ml, 3.0 mmol) for 6 hours at 150° C. After working-up and chromatographic purification (silica gel, hexane/ethyl acetate (0-30% ethyl acetate)), 247 mg (40% of theory) of the product is obtained.

NMR (300 MHz, DMSO-d₆): δ2.80 (s, 3H), 3.05 (d, 1H), 3.54 (d, 1H), 3.60 (s, 3H), 6.60 (s, 1H), 6.80-6.95 (m, 2H), 7.10 (dd, 1H), 7.24 (td, 1H), 7.54 (dd, 1H), 7.81 (dd, 1H), 8.20 (s, 1H), 9.32 (s, 1H).

### b) 2-(8-Fluoro-2-methylquinazolin-5-ylamino)-3-trifluoromethylchroman-3-ol

Analogously to Example 1, a solution that consists of 1,1,1-trifluoro-3-(8-fluoro-2-methyl-quinazolin-5-ylimino)-2-(2-methoxybenzyl)-propan-2-ol (90 mg, 0.22 mmol) in 2.0 ml of dichloromethane is mixed at room temperature with 2.2 ml (10 equivalents) of a 1 M solution of boron tribromide in dichloromethane. After 4 hours, as described in Example 1, it is worked up, and the residue is purified by means of preparative thin-layer chromatography (hexane/ethyl acetate 1:1). 55 mg (63% of theory) of the product is obtained.

NMR (300 MHz, DMSO-d₆): δ2.78 (s, 3H), 3.13 (d, 1H), 3.37 (d, 1H), 5.84 (d, 1H), 6.83-6.87 (m, 2H), 6.94 (t, 1H), 7.08-7.23 (m, 4H), 7.69 (dd, 1H), 9.68 (s, 1H).

### Example 5

### 5-(3-Hydroxy-3-trifluoromethylchroman-2-ylamino)-3H-isobenzofuran-1-one

### a) 5-[3,3,3-Trifluoro-2-hydroxy-(2methoxybenzyl)-propylidenamino]-3H-isobenzofuran-1-one

Analogously to Example 1, 3,3,3-trifluoro-2-hydroxy-2-(2-methoxybenzyl)-propionaldehyde (103 mg, 0.41 mmol) is reacted with 5-amino-3H-isobenzofuran-1-one (93 mg, 0.62 mmol) in 6 ml of xylene in the presence of titanium(IV)ethylate (0.18 ml, 0.83 mmol) for 6 hours at 150° C. After working-up and chromatographic purification (silica gel, hexane/ethyl acetate (0-60% ethyl acetate)), 76 mg (48% of theory) of the product is obtained.

NMR (300 MHz, DMSO-d₆): δ3.15 (d, 1H), 3.43 (d, 1H), 3.68 (s, 3H), 5.38 (s, 2H), 6.42 (s, 1H), 6.85-7.00 (m, 3H), 7.08 (s, 1H), 7.20-7.30 (m, 2H), 7.82 (d, 1H), 7.95 (s, 1H).

### b) 5-(3-Hydroxy-3-trifluoromethylchroman-2-ylamino)-3H-isobenzofuran-1-one

Analogously to Example 1, a solution that consists of 5-[3,3,3-trifluoro-2-hydroxy-(2-methoxybenzyl)-propylidenamino]-3H-isobenzofuran-1-one (70 mg, 0.18 mmol) in 2.0 ml of dichloromethane is mixed at room temperature with 1.86 ml (10 equivalents) of a 1 M solution of boron tribromide in dichloromethane. After 4 hours, as described in Example 1, it is worked up, and the residue is purified by means of chromatography (hexane/ethyl acetate 1:1). 37 mg (55% of theory) of the product is obtained.

NMR (300 MHz, MeOD): δ3.20 (d, 1H), 3.40 (d, 1H), 5.30 (s, 2H), 5.75 (s, 1H), 6.82 (d, 1H), 6.94 (t, 1H), 7.05-7.20 (m, 4H), 7.70 (d, 1H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102004025791.4, filed May 19, 2004, and U.S. Provisional Application Ser. No. 60/576,408, filed Jun. 3, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula I 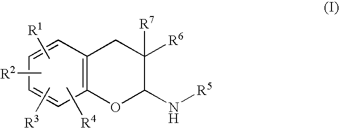
in which
R¹, R² and R³, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a C₁-C₅-perfluoroalkyl group, a cyano group, a nitro group, or a group NR⁸R⁹,
whereby R⁸ and R⁹, independently of one another, can be a hydrogen atom, a (C₁-C₅)-alkyl group, a CO[O(C₁-C₅)]-alkyl group or a (CO)—C₁-C₅-alkyl radical,
R⁴ means a hydrogen atom, a hydroxy group, a halogen atom, a cyano group, an optionally substituted (C₁-C₁₀)-alkyl group, a (C₁-C₁₀)-alkoxy group, or R¹ and R² or R² and R³ or R³ and R⁴ together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—,
whereby n=1 or 2, and the terminal oxygen atoms, nitrogen atoms and/or carbon atoms are linked to directly adjacent ring-carbon atoms,
R⁵ means a C₁-C₁₀-alkyl group, which optionally can be substituted by a group that is selected from 1-3 hydroxy groups, halogen atom, or 1-3 (C₁-C₅)— alkoxy groups,
an optionally substituted (C₃-C₇)-cycloalkyl group,
an optionally substituted heterocyclyl group,
an optionally substituted aryl group,
a monocyclic or bicyclic aromatic, partially aromatic or non-aromatic ring system, which optionally contains 1-3 nitrogen atoms, 1-2 oxygen atoms and/or 1-2 sulfur atoms and/or 1-2 keto groups, and optionally is substituted in one or more places, independently of one another, by a radical that is selected from the group of carbonyl, halogen atom, hydroxy group, or (C₁-C₅)-alkyl group, which optionally can be substituted by 1-3 hydroxy groups, 1-3 (C₁-C₅)alkoxy groups and/or 1-3 COOR¹⁰ groups,
(C₁-C₅)alkoxy group, (C₁-C₅)-alkylthio group, (C₁-C₅)-perfluoralkyl group, cyano group, nitro group, or the radical NR¹¹R⁹
whereby R⁸ and R⁹, independently of one another, mean hydrogen, C₁-C₅-alkyl, a CO[O(C₁-C₅)]-alkyl group or (CO)—C₁-C₅-alkyl group,
or the group COOR¹⁰,
whereby R¹⁰ means hydrogen or a C₁-C₅-alkyl group,
or the group (CO)NR¹¹R¹²,
whereby R¹¹ and R¹², independently of one another, mean hydrogen or a C₁-C₅-alkyl group,
or a (C₁-C₅-alkylene)-O—(CO)—(C₁-C₅)alkyl group,
whereby this ring system can be linked via any position to the amine of the chromanol system and optionally can be hydrogenated in one or more places,
R⁶ means a (C₁-C₅)-alkyl group or an optionally partially or completely fluorinated (C₁-C₅)-alkyl group, a (C₃-C₇)cycloalkyl group, a (C₁-C₈)alkyl(C₃-C₇)cycloalkyl group, a (C₂-C₈)alkenyl(C₃-C₇)cycloalkyl group, a heterocyclyl group, a (C₁-C₈)alkylheterocyclyl group, a (C₂-C₈)-alkenylheterocyclyl group, a (C₂-C₈)alkinylaryl group, an aryl group, a (C₁-C₈)alkylaryl group, a (C₂-C₈)alkenylaryl group; a monocyclic or bicyclic heteroaryl group, (C₁-C₈)alkylheteroaryl group or (C₂-C₈)alkenylheteroaryl group that is optionally substituted by 1-2 keto groups, 1-2 (C₁-C₅)-alkyl groups, 1-2 (C₁-C₅)-alkoxy groups, 1-3 halogen atoms, or 1-2 exomethylene groups and/or that contains 1-3 nitrogen atoms and/or 1-2 oxygen atoms and/or 1-2 sulfur atoms,
R⁷ means a hydroxy group or a group OR¹³
whereby R¹³ means a C₁-C₁₀-alkyl group,
as well as their racemates or separately present stereoisomers, and optionally their physiologically compatible salts.

2. Compounds of general formula I according to claim 1, in which
R¹, R² and R³, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a C₁-C₅-perfluoroalkyl group, a cyano group, a nitro group, or a group NR⁸R⁹,
whereby R⁸ and R⁹, independently of one another, can be a hydrogen atom, a (C₁-C₅)-alkyl group, a CO[O(C₁-C₅)]-alkyl group or a (CO)—C₁-C₅-alkyl radical,
R⁴ means a hydrogen atom, a hydroxy group, a halogen atom, a cyano group, an optionally substituted (C₁-C₁₀)-alkyl group, a (C₁-C₁₀)-alkoxy group or R¹ and R² or R² and R³ or R³ and R⁴ together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—,
whereby n=1 or 2, and the terminal oxygen atoms, nitrogen atoms and/or carbon atoms are linked to directly adjacent ring-carbon atoms,
R⁵ means a monocyclic or bicyclic aromatic, partially aromatic or non-aromatic ring system, which optionally contains 1-3 nitrogen atoms, 1-2 oxygen atoms, and/or 1-2 sulfur atoms and/or 1-2 keto groups, and optionally is substituted in one or more places, independently of one another, by a radical that is selected from the group of carbonyl, halogen atom, hydroxy group, (C₁-C₅)-alkyl group, which optionally can be substituted by 1-3 hydroxy groups, 1-3 (C₁-C₅)alkoxy groups and/or 1-3 COOR¹⁰ groups,
(C₁-C₅)alkoxy group, (C₁-C₅)-alkylthio group, (C₁-C₅)-perfluoroalkyl group, cyano group, nitro group, or the radical NR⁸R⁹,
whereby R⁸ and R⁹, independently of one another, mean hydrogen, C₁-C₅-alkyl, a CO[O(C₁-C₅)]-alkyl group or (CO)—C₁-C₅-alkyl group,
or the group COOR¹⁰,
whereby R¹⁰ means hydrogen or a C₁-C₅-alkyl group,
or the group (CO)NR¹¹R¹²,
whereby R¹¹ and R², independently of one another, mean hydrogen or a C₁-C₅-alkyl group,
or a (C₁-C₅-alkylene)-O—(CO)—(C₁-C₅)alkyl group,
whereby this ring system can be linked via any position with the amine of the chromanol system and optionally can be hydrogenated at one or more locations,
R⁶ means a (C₁-C₅)-alkyl group or an optionally partially or completely fluorinated (C₁-C₅)-alkyl group, a (C₃-C₇)cycloalkyl group, a (C₁-C₈)alkyl(C₃-C₇)cycloalkyl group, a (C₂-C₈)alkenyl(C₃-C₇)cycloalkyl group, a heterocyclyl group, a (C₁-C₈)alkylheterocyclyl group, a (C₂-C₈)-alkenylheterocyclyl group, a (C₂-C₈)alkinylaryl group, an aryl group, a (C₁-C₈)alkylaryl group, a (C₂-C₈)alkenylaryl group, a monocyclic or bicyclic heteroaryl group, (C₁-C₈)alkylheteroaryl group or (C₂-C₈)alkenylheteroaryl group that is optionally substituted by 1-2 keto groups, 1-2 (C₁-C₅)-alkyl groups, 1-2 (C₁-C₅)-alkoxy groups, 1-3 halogen atoms, or 1-2 exomethylene groups and/or that contains 1-3 nitrogen atoms and/or 1-2 oxygen atoms and/or 1-2 sulfur atoms,
R⁷ means a hydroxy group or a group OR¹³,
whereby R¹³ means a C₁-C₁₀-alkyl group,
as well as their racemates or separately present stereoisomers, and optionally their physiologically compatible salts.

3. Compounds according to claim 1, in which
R¹, R² and R³, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a C₁-C₅-perfluoralkyl group, a cyano group, a nitro group, or a group NR⁸R⁹,
whereby R⁸ and R⁹, independently of one another, can be a hydrogen atom, a (C₁-C₅)-alkyl group, a CO[O(C₁-C₅)]-alkyl group or a (CO)—C₁-C₅-alkyl radical,
R⁴ means a hydrogen atom, a hydroxy group, a halogen atom, a cyano group, an optionally substituted (C₁-C₁₀)-alkyl group, a (C₁-C₁₀)-alkoxy group or R¹ and R² or R² and R³ or R³ and R⁴ together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, —NH—N═CH—,
whereby n=1 or 2, and the terminal oxygen atoms, nitrogen atoms and/or carbon atoms are linked to directly adjacent ring-carbon atoms,
R⁵ means a monocyclic or bicyclic aromatic, partially aromatic or non-aromatic ring system, which optionally contains 1-3 nitrogen atoms, 1-2 oxygen atoms and/or 1-2 sulfur atoms and/or 1-2 keto groups, and optionally is substituted in one or more places, independently of one another, by a radical that is selected from the group of carbonyl, halogen atom, hydroxy group, (C₁-C₅)-alkyl group, which optionally can be substituted by 1-3 hydroxy groups, 1-3 (C₁-C₅)alkoxy groups and/or 1-3 COOR¹⁰ groups,
(C₁-C₅)alkoxy group, (C₁-C₅)-alkylthio group, (C₁-C₅)-perfluoroalkyl group, cyano group, nitro group, or the radical NR⁸R⁹,
whereby R⁸ and R⁹, independently of one another, mean hydrogen, C₁-C₅-alkyl, a CO[O(C₁-C₅)]-alkyl group or a (CO)—C₁-C₅-alkyl group,
or the group COOR¹⁰,
whereby R¹⁰ means hydrogen or a C₁-C₅-alkyl group,
or the group (CO)NR¹¹R¹²,
whereby R¹¹ and R², independently of one another, mean hydrogen or a C₁-C₅-alkyl group,
or a (C₁-C₅-alkylene)-O—(CO)—(C₁-C₅)alkyl group,
whereby this ring system can be linked via any position to the amine of the chromanol system and optionally can be hydrogenated on one or more locations,
R⁶ means an optionally partially or completely fluorinated (C₁-C₅)-alkyl group,
R⁷ means a hydroxy group or a group OR
whereby R¹³ means a C₁-C₁₀-alkyl group,
as well as their racemates or separately present stereoisomers, and optionally their physiologically compatible salts.

4. Stereoisomers according to claim 1, in which R⁵ means optionally substituted phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that is linked via any position as well as their physiologically compatible salts.

5. Stereoisomers according to claim 1, in which R⁵ is substituted by one or more substituents that are selected from the group of (C₁-C₅)-alkyl group, (C₁-C₅)-alkoxy group, halogen atom, a keto-oxygen atom or a hydroxy group.

6. Compounds according to claim 1, in which R⁵ means a phenyl ring, which optionally can be substituted in one or more places by a radical that is selected from the group C₁-C₅-alkyl, C₁-C₅-alkoxy, C₁-C₅-alkylthio, C₁-C₅-perfluoroalkyl, halogen, hydroxy, cyano, nitro, —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, —NH—N═CH—,
whereby n=1 or 2 and the terminal oxygen atoms, nitrogen atoms and/or carbon atoms are linked to directly adjacent ring-carbon atoms,
or NR⁸R⁹,
whereby R⁸ and R⁹, independently of one another, can be a hydrogen atom, a C₁-C₅-alkyl group or a (CO)—C₁-C₅-alkyl group.

7. Process for the production of compounds of general formula I, characterized in that compounds of general formula II 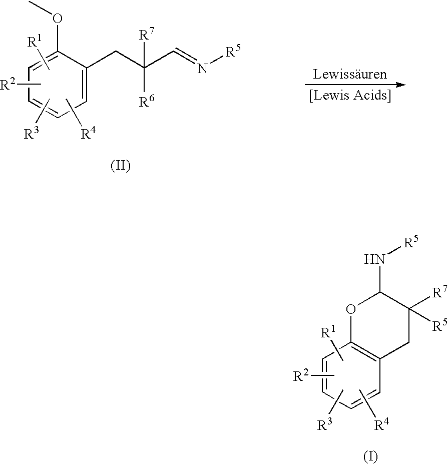
are reacted to form compounds of general formula (I) by reaction with Lewis acids at −70° C. to +30° C.

8. Use of the compounds according to claim 1 for the production of pharmaceutical agents.

9. Use of the compounds according to claim 1 for the production of pharmaceutical agents for treating inflammatory diseases.
